(19) United States
(12) Patent Application Publication
(10) Pub. No.: US 2024/0117404 A1
(43) Pub. Date: Apr. 11, 2024

(54) Automatic Phagogram

(71) Applicant: INTELIPHAGE SRL, Mont-Saint-Guibert (BE)

(72) Inventors: Jehan LIENART VAN LIDTH DE JEUDE, Pommerloch (LU); Johan QUINTENS, Hoegaarden (BE); Bob BLASDEL, Etterbeek (BE)

(73) Assignee: INTELIPHAGE SRL, Mont-Saint-Guibert (BE)

(21) Appl. No.: 18/275,860

(22) PCT Filed: Feb. 4, 2022
(86) PCT No.: PCT/EP2022/052743

§ 371 Date: Aug. 4, 2023

(30) Foreign Application Priority Data
Feb. 4, 2021 (BE) BE2021/5084

(51) Int. Cl. C12Q 1/18; B01L 3/00

(52) U.S. Cl. CPC C12Q 1/18; B01L 3/5085; B01L 2200/0647; B01L 2200/16; B01L 2300/0829

(57) ABSTRACT

A system for measuring the sensitivity of a bacterium to a plurality of phages, the system comprising: a microwell plate comprising a plurality of wells, each of said plurality of wells being configured to contain one of a plurality of phages, wherein said microwell plate is configured to have a sample comprising said bacterium dispensed into said plurality of wells; at least one reagent and/or additive, configured to be added to mixtures of said sample comprising said bacterium with a phage of said plurality of phages; and a control unit configured to interpret and present the results of measurements in the form of a phagogram, said measurements being provided by at least one analytical device, said at least one analytical device being configured to measure the interaction of each of said plurality of phages with said bacterium. The invention also relates to a corresponding method for use in same.

### FIELD OF THE INVENTION

The present invention relates to a system for measuring the sensitivity of a bacterium to a plurality of phages and for reporting said sensitivities and possible lytic effects in the form of an automatized phagogram. The invention also relates to a corresponding method for use in same.

### BACKGROUND

Antimicrobial resistance (AMR) is a growing problem worldwide that poses a significant threat to human and animal health, in both modern veterinary and clinical environments. Today, pathogenic bacteria resistant to virtually all commercially available antibiotics have emerged and there are currently few molecules in the pipeline for the development of new classes of antibiotics. The article by Marston et al., “Antimicrobial Resistance”, JAMA 316(11) doi: 10.1001/jama.2016.11764 identifies factors associated with AMR. The World Health Organisation (WHO) has named antibiotic resistance one of the biggest threats to global health, food security, and development and estimates that at least 700,000 people die each year due to drug-resistant diseases (WHO, Joint News Release, April 2019). Initially limited in some geographical regions, AMR now represents a challenge for health specialists worldwide, from clinical microbiologists and infectious control diseases specialists to veterinarians. The recent description of patients harbouring multi-resistant bacteria underlines the clinical context but also the potential consequence of such bacterial threat.

The advantages of the use of bacteriophages as antimicrobial agents over antibiotics have been known for some time. One significant advantage of phages is their specificity to species (or strains thereof) and genera, which allows phage treatment to remove targeted organisms without disturbing the local microbiome, reducing the chances of opportunistic infections. Phages need to be administered for a relative short duration of time and can potentially be used in conjunction with antibiotics to delay resistance. An elaborated overview of the advantages of the use of phages as antibacterial therapeutics as well as some of the limitations and challenges are presented in Nikolich et al. “Bacteriophage Therapy: Developments and Directions” Antibiotics, 2020, 9, 135, doi:10.3390/antibiotics9030135, the Introduction Part of which is herein incorporated by reference.

Nevertheless, the mostly species-specific or strain-specific behaviour of phages and their particularly narrow host range can also be interpreted as a significant disadvantage relative to antibiotics.

To overcome this inherent weakness, a popular general approach for phage therapy focuses on making static mixes or “cocktails” of multiple phage components that are selected to infect the largest possible proportion of strains currently infecting patients and/or animals, and/or environments. However, any static cocktail of phages will only be capable of effectively treating a subset of patients and those cocktails will rapidly lose relevance as bacterial populations continue to evolve. The reader will find further information in the article by Rohde et al., “Expert Opinion on Three Phage Therapy Related Topics: Bacterial Phage Resistance, Phage Training and Prophages in Bacterial Production Strains”, Viruses 2018, 10(4), 178; https://doi.org/10.3390/v10040178.

Alternatively, personalized phage therapy strategies seek to assemble a larger library of phages than could be feasibly assembled into a cocktail and to only treat patients with phages that have been identified to be active on their strains, thereby producing a therapeutic tailored to an individual patient's infection. As such, a therapeutic is developed in direct response to an individual patient's infection, the therapeutic containing a phage or phages having lytic activity against the pathogen in the infection. The article by Pirnay et al., “The Magistral Phage”, Viruses 2018, 10(2), 64; doi: 10.3390/v10020064 discusses the implementation of a legal and regulatory framework for the magisterial preparation of tailor-made phage medicines.

A further disadvantage is that current diagnostic methodologies for in vitro phage susceptibility testing are adapted for an academic laboratory environment rather than the kind of clinical testing environment that can be made broadly accessible to patients.

Konopacki et al. disclose in “PhageScore: A simple method for comparative evaluation of bacteriophages lytic activity”, Biochemical Engineering Journal, 161,2020, doi.org/10.1016/j.bej.2020.107652, a method for bacteriophage efficiency evaluation by measuring bacteria growth curves with OD₆₀₀ₙₘ for a period of at least 4 hours. Xie et al. disclose in “Development and Validation of a Microtiter Plate-based Assay for Determination of Bacteriophage Host Range and Virulence”, Viruses 10, 189, doi:10.3390/v10040189, a study for measuring the phage host range and virulence for a collection of 15 Salmonella phages against a panel of Salmonella strains by use of a spot assay as well as by microtiter plate liquid assay at two initial phage concentrations with OD₅₅₀ₙₘ for a period of at least 12 hours.

In both studies, testing the susceptibility of a bacterial pathogenic sample to a library of phages can be considered time consuming and requires a commercially infeasible amount of manual operations by qualified staff.

Indeed, currently used techniques require an exponentially growing culture obtained from a pure culture isolate that typically requires a day to generate, require at least an additional day to conduct, and moreover require highly trained staff, the latter being inaccessible in a professional clinical environment.

By the time it is identified, an infection caused by bacterial pathogens can become risky and even life-threatening in a matter of days, or even a few hours. Hence, a need exists for a method of rapidly ascertaining whether a particular strain of bacteria is susceptible to a potentially therapeutic bacteriophage.

WO 99/57304 relates to an assay for identifying bacteria present in a sample and a method for treating a bacterial infection. The protocol disclosed in the document includes growing a bacterial isolate overnight, dispensing samples on a microtiter plate and incubate the plate overnight. Interaction of the phage and bacteria is thereby determined by measuring the extent of bacterial growth following incubation with the phage.

WO 2004/041156 relates to a method for identifying bacteria and for selecting a therapeutic bacteriophage. Expression of a reporter molecule indicates whether a bacteriophage is capable of infecting a bacterial cell in a sample. Samples collected from a patient are incubated for 4 to 12 hours.

WO2017223101 relates to a method of compounding a phage cocktail directed against a bacterial pathogen, the method comprising the steps of constructing a bacterial diversity set comprising diverse strains of the same bacterial species, and subsequently constructing an archival phage library and a working phage library, wherein the latter is screened for a delay in bacterial growth and/or a lack of appearance of phage-resistant bacterial growth.

Fischer et al. disclose in “Microplate-Test for the Rapid Determination of Bacteriophage-Susceptibility of Campylobacter Isolates—Development and Validation”, PLoS ONE 8(1), doi:10.1371/journal.pone.0053899, a microplate susceptibility test for the rapid determination of bacteriophage-susceptibility of Campylobacter Isolates, comparing the simplified test with the conventional agar overlay plate assay. Plates were incubated under microaerobic conditions after solidification for 18 h.

There is therefore a need for a rapid and easy determination of bacteriophages suitable for treating a particular bacterial infection.

### SUMMARY OF THE INVENTION

According to an aspect of the present invention, there is provided a system for measuring the sensitivity of a bacterium, the bacterium being preferably a bacterial pathogen, to a plurality of phages, the system comprising:
a microwell plate comprising a plurality of wells, wherein each of said plurality of wells is configured to contain one of a plurality of phages, wherein said microwell plate is further configured to have a sample comprising said bacterium dispensed into each of said plurality of wells;
at least one reagent and/or additive, configured to be added to at least one mixture of said sample comprising said bacterium and a phage of said plurality of phages; and
a control unit configured to interpret and present the results of measurements in the form of a phagogram, said measurements being provided by at least one analytical device, said at least one analytical device being configured to measure the interaction of each of said plurality of phages with said bacterial pathogen.

More in particular, there is provided a system for measuring the sensitivity of a bacterium, the bacterium being preferably a bacterial pathogen, to a plurality of phages, the system comprising:
a microwell plate comprising a plurality of wells for each containing one of a plurality of phages and for having a sample comprising said bacterium dispensed therein;
at least one reagent and/or additive for being added to at least one mixture of said sample comprising said bacterium and a phage of said plurality of phages; and
a control unit configured to interpret and present the results of measurements in the form of a phagogram, said measurements being provided by at least one analytical device, said at least one analytical device being configured to measure the interaction of each of said plurality of phages with said bacterial pathogen.

It is an advantage of the present invention that the sensitivity of a bacterium can be tested against a plurality of bacteriophages by use of one single microwell plate or defined array of plates. As such, a phage interacting with said bacterium can be identified in a relative rapid way. The microwell plate is by preference disposable and is preferably provided with a set or plurality of bacteriophages which are selected due to their capacity to cause cell inactivation, and preferably cell lysis, and preferably phage propagation, to a specific species or genera of a bacterium or bacterial pathogen and/or strains thereof, or an assembled set of specific species or genera of bacteria or bacterial pathogens and strains thereof. Advantageously, the effects of said selection of bacteriophages on the bacterium can be detected by use of a single experimental technique, by means of a single analytical device and/or software package.

It is a further advantage that the measurements are represented in the form of an automatic phagogram, thereby offering a direct summary of phage-bacterium interaction and allowing identification of the bacterial species and/or of the phage(s) which cause cell inactivation, cell lysis and preferably phage propagation, in said bacterial species.

It is a further advantage of the present invention that such a phagogram can be obtained within a relative short period of time, particularly when compared to the currently existing methods for obtaining such information. Indeed, the time needed between collecting a workable sample from a patient and retrieving the phagogram may even be reduced to a few hours, and, in the case a native sample may be used, may even be around 1-2 to 4 hours.

In embodiments of the system according to the present invention, said sample is a native sample from a patient.

It is an advantage of this embodiment that the system and method as described herein can be applied directly in combination with a native sample, said native sample being typically directly obtained from a patient, animal, infectious herd, or environment. Due to the specificity in phage-bacterium interaction, there may be no need to isolate and purify the bacterium or bacterial pathogen of interest. It may then be assumed that a plurality of phages that are selected for their capacity to interact with a bacterium of interest in a sample will not react with unrelated bacteria that may be present in that sample. Likewise, there may be no need to obtain a pure culture isolate of the bacterial pathogen of interest from the patient sample and/or to incubate the sample collected from a patient in order to grow a number of colony-forming units (CFU) which is considered to be sufficient for contacting with a plurality of bacteriophages. As such, crucial time is saved in the search for the identification of the source of a bacterial infection as well as identification of phages interacting with and/or causing cell lysis of the bacterium or bacterial pathogen.

As a comparison, protocols for obtaining an antibiogram have been developed for some time and include collecting patient samples in order to generate pure culture isolates of the infectious agent. This procedure typically requires an 18-hour waiting period while colonies of the pure culture isolate grow. Only then can modern antibiograms produce an actionable diagnosis of the antibiotic sensitivity of the isolate—typically within an hour.

In embodiments of the system according to the present invention, the microwell plate is disposable.

As used herein, the term “disposable” refers to being designed to be used once and then being discarded. It is an advantage of these embodiments that microplates can be used which are pre-prepared and sealed and ready for use at the point of use. Such microplates may be provided with a protocol for use, which may include a computer program product or script for a processor in order to provide each well of the microplate with the right (amount of) reagents and/or additives. Such a microplate may be a standardized plate, provided with a plurality of bacteriophages capable of provoking cell lysis in a specific bacterium, and its strains, and may be configured to investigate the presence of said specific bacterium in a sample.

In embodiments of the system according to the present invention, the plurality of wells is further configured to contain at least one selection of phages, wherein each of said selection of phages is selected by the capacity to interact with and/or inactivate a single specific bacterial species, and strains thereof.

In embodiments of the system according to the present invention, the plurality of wells relates to a plurality of wells for containing at least one selection of phages, wherein each of said selection of phages is selected by the capacity to interact with and/or inactivate a single specific bacterial species, and strains thereof.

It is an advantage of these embodiments that by combining phages on a single microplate that target different strains and/or different receptor types of a specific bacterial species, typically a specific bacterial pathogen, a characterization of the species or pathogen may be obtained, which allows for the development of a therapy or phage cocktail which strongly decreases the risk of the bacterial pathogen of developing phage resistance. Preferably, a selection comprises a combination of phages so that interaction with a maximal number of strains of the bacterial species can be investigated.

In embodiments of the system according to the present invention, said at least one reagent and/or additive comprises at least one of the following: a primer molecule, a DNA polymerase, a nucleoside triphosphate (dNTP), a molecular beacon probe capable to fluoresce, a buffer, and a qPCR-amplification compatible solvent and/or enzyme. Furthermore, said at least one analytical device is configured to measure the interaction of each of said plurality of phages with said bacterium by measuring the abundance and/or differential concentration of phage DNA after co-incubation of said bacterium with each of said plurality of phages by use of quantitative polymerase chain reaction. Said at least one analytical device is thereby preferably configured to measure the sensitivity of said bacterium to each of said plurality of phages by measuring the abundance and/or differential concentration of phage DNA after co-incubation of said bacterium with each of said plurality of phages by use of quantitative polymerase chain reaction.

It is an advantage of these embodiments that bacterium—phage interactions may be measured in a relatively quick manner, as compared to currently available methods. Advantageously, several samples may be measured simultaneously. A qPCR measurement of a microplate containing bacterium—phage mixtures may be measured in less than 2 hours. Furthermore, detailed information about the phage proliferation can be calculated where in other detection methods information is often limited to bacterial cell death. Such information can permit to quantify the virulence of a certain phage compared to others.

In embodiments of the system according to the present invention, said at least one reagent and/or additive comprises at least one of the following: a Luciferin/Luciferase complex configured to allow detection of bacterial cell lysis through depletion of ATP released by phage-mediated lysis and concomitant fluorescein light emission, and a Luciferin/Luciferase complex compatible solvent and/or enzyme. Furthermore, said at least one analytical device is configured to measure the interaction of each of said plurality of phages with said bacterium by measuring said light emission.

It is an advantage of these embodiments that bacterium—phage interactions may be measured in a relatively quick manner, as compared to currently available methods. Advantageously, several samples may be measured simultaneously. A bioluminescence measurement of a microplate containing bacterium—phage mixtures with a luciferase/luciferin complex may be measured within minutes, typically in about 1-2 minutes, even in less than a minute. Furthermore, measuring bioluminescence by adding a luciferase/luciferin complex allows for accurate results, the technique is sensitive and fairly easy to execute.

In embodiments of the system according to the present invention, said control unit is furthermore configured to provide a composition of phages on the basis of said phagogram, wherein said composition of phages interact with a maximal number of different binding receptor types of the bacterium or bacterial species of interest. Preferably, said composition does not contain more than three different phage species. Preferably, each phage species interacts with a different type of binding receptor of the bacterial species of interest.

It is an advantage of these embodiments that a composition of phages may be obtained which may be suitable for the treatment of a bacterial infection caused by said bacterium or bacterial pathogen. Bacteria may have one or more specific receptor motifs arrayed on their surface, which mediate their binding and adsorption cascades. These receptor motifs can be found in cell surface molecules including but not limited to sugars, glycoproteins, proteins, lipids and lipoproteins. Phages may be capable of detecting one or more specific receptor motifs and can therefore be lumped together into ‘receptor groups’ according to the specific molecules that their receptor motifs are found on. It is a further advantage of these embodiments that one single system is needed to obtain a possible companion diagnostic for a bacterial infection, caused by a bacterial pathogen in a sample, which was provided to the system.

In embodiments of the system according to the present invention, the control unit is furthermore connected to a central database and reports results, including the obtained phagograms, thereto.

It is an advantage of these embodiments that the results of a high number of phagograms performed in diverse clinical environments enable the identification of the host ranges of phages on a large and relevant array of bacteria. As such, trends in host range by region, over time, by health system, and by indication may be differentiated, allowing to identify outbreaks of diseases. Such outbreaks may be identified by “phage type” or the specific array of phages that can infect a specific strain that forms a unique and identifiable signature and which may be tracked by the phagogram.

According to an aspect of the present invention, there is provided a method for measuring the sensitivity of a bacterium to a plurality of phages, the method having the following steps:
Providing a first composition comprising said bacterium and a first microwell plate comprising a plurality of wells, each of said plurality of wells containing one of said plurality of phages;
Dispensing a first sample of said first composition in at least one well of said plurality of wells;
For each of said at least one well, allowing incubation of the mixture of said first sample comprising said bacterium and a bacteriophage contained in the well for the duration of a first period of time;
Adding at least one reagent and/or additive to each mixture, and
Transporting to and introducing at least one mixture in an analytical device and measuring the bacterium-phage interaction of said at least one mixture by said analytical device; and
Interpreting and presenting the measurements by said analytical device in the form of a phagogram by a control unit.

In embodiments of the method according to the present invention, the method further comprises the step of transferring a second sample of said mixture to a second microwell plate, prior to adding said at least one reagent, wherein said second microwell plate is configured to be compatible with the analytical device.

It is an advantage of these embodiments that the use of a second microwell plate allows the utilization of microplates which are configured to be compatible with a specific analytical device. Furthermore, said second microplates can be configured to facilitate the addition of extra reagents and/or additives prior to analysis by the analytical device.

In embodiments of the method according to the present invention, said first composition is a native sample from a patient.

In embodiments of the method according to the present invention, measuring the bacterium-phage interaction by said analytical device implies measuring the abundance and/or differential concentration of phage DNA by use of quantitative polymerase chain reaction.

In embodiments of the method according to the present invention, measuring the bacterium-phage interaction by said analytical device implies detecting cell lysis by measuring bioluminescence through addition of luciferase activity.

In embodiments of the method according to the present invention, the method further comprises the step of selecting a composition of phages on the basis of said phagogram, wherein the phages of said composition of phages interact with a maximal number of different binding receptor types of said bacterium.

According to an aspect of the present invention, there is provided a microwell plate comprising a plurality of wells for each containing one of a plurality of phages, for use in the system and method as described herein.

According to an aspect of the present invention, there is provided a computer program product comprising code means configured to cause a processor to perform the functions of said system and method.

### BRIEF DESCRIPTION OF THE FIGURES

These and other features and advantages of embodiments of the present invention will now be described in more detail with reference to the accompanying drawings, in which:

FIG. 1 schematically illustrates a system and a method for measuring the sensitivity of a bacterium to a plurality of phages according to embodiments of the invention.

FIGS. 2a-2c schematically illustrate a method for measuring the sensitivity of a bacterium to a plurality of phages and for the selection of a composition of phages on the basis of a phagogram.

### DETAILED DESCRIPTION OF EMBODIMENTS

### Definitions

The term “bacteriophage” or “phage” refers herein to any virus or virus-like entity whose host is a bacterium.

Phages form a highly diverse group of viruses.

The term “Phagogram” refers herein to a collection of data, preferably in the form of a table, summarizing the effect of a plurality of individual bacteriophages on either an individual bacterial pathogen or mixed sample environment. In some respects equivalent to an antibiogram, the phagogram gives a profile of an organism's resistance or susceptibility to a panel of phages.

The term “native sample” refers to a sample containing a bacterium, typically a pathogenic bacterium or an infectious bacterial pathogen, said sample being collected from a human, animal (s), or environment during the course of pathogenesis or gradual progression of a specific disease. No isolation and monoculture of a specific pathogen of the sample has been performed.

Herein, a bacterium is considered sensitive to a phage when interaction with said phage leads to the death or inactivation of said bacterium. Preferably, this interaction also leads to the propagation of said phage within said bacterium leading to cell lysis and the release of phage progeny.

The term “receptor” refers to a chemical group or molecule (moiety) such as a protein or sugar on the cell surface or in the cell interior that has an affinity for a specific chemical group, molecule, or virus. A receptor is a cellular chemical moiety recognized by a viral particle that is involved in specifically inducing adsorption.

Applications and/or goals of the system and method described herein include
the identification of a bacterium, typically a bacterial pathogen, in a sample, the sample being a native sample from a patient, or a pure culture isolate thereof,
the identification of any phages that cause cell inactivation, and more preferably cell lysis and even more preferably phage propagation, in said bacterium in said sample, and/or
the development of a composition or cocktail of phages for the purpose of treating a bacterial infection caused by said bacterium.

Although reference may be made herein to a bacterial pathogen or a pathogenic bacterium, terms which are used interchangeably herein, the system and method as described herein may be applied on any bacterium or mixture of bacteria that are defined taxonomically at the variant, strain, species, Generic, or Family levels.

In specific cases, a patient may have already been diagnosed with a bacterial infection, wherein a specific species or genus of bacterial pathogen may be suspected to have caused the infection. A selection of bacteriophages may then be contacted with the bacterial pathogen in a sample, or a pure culture isolate thereof, for the purpose of studying and measuring the effectiveness of each individual phage of the selection against said bacterial pathogen. Each of the bacteriophages in the selection has preferably been selected on the basis of its capacity to affect at least one strain of the suspected pathogen species.

In embodiments according to the invention, bacteriophages may be selected because of their capacity to be effective against a specific bacterial pathogen species. A bacteriophage is thereby considered effective when it causes cell lysis in said species, and preferably propagates in the bacterial host, replicating its bacteriophage DNA in the host cell. In particular embodiments, bacteriophages may be selected because of their capacity to be effective against one of a variety of strains of said bacterial pathogen.

An application of the system may then be to verify the assumed bacterial identity and/or to provide for a Phagogram supplying results regarding the effectiveness of a selection of bacteriophages against a bacterial pathogen having one or more strains. It is an advantage that the system and method as described herein provide for a rapid handling and procedure so that no valuable time is lost in search for an effective treatment of the patient.

Phages do engender resistance in bacteria similarly to antibiotics, with resistant mutants being often deficient in the binding receptor used by the phage to identify target bacteria. For pathogenic bacteria, these binding receptors are often also virulence factors involved in a critical aspect of pathogenesis.

In embodiments according to the invention, bacteriophages may be further selected due to their capacity to collectively employ a maximal number of different receptors of the bacterial pathogen. As such, multiple virulence factors may be targeted simultaneously.

A typical application of the system for the design of a treatment involves measuring the effectiveness of a selection of phages against a bacterial pathogen in a sample, wherein said phages target a maximal number of host receptors and at least one of a variety of strains of said bacterial pathogen, and wherein said sample is a native sample from a patient, or a pure culture isolate thereof.

The invention is based on the principle that the highly specific interaction between phages and bacteria can be determined through the measurement of various consequences of phage expression when in contact with a bacterial sample. The effect of the phage on a host bacterium will vary from complete lysis of the bacterium associated with phage propagation to no noticeable effect. By identifying a phage which is efficient in causing cell death in such a particular host bacterium species or strain, preferably accompanied by phage propagation, a treatment can be developed for a patient suffering a bacterial infection.

The inventors have found that such phage expression can be measured while the phage is at different stages of its cycle.

It is known that Bacteriophages propagate by the following steps
(1) Attaching to specific susceptible host cells,
(2) Replacing the host metabolism with a phage metabolism, arresting cell growth,
(3) Replicating the bacteriophage DNA,
(4) Packaging this Bacteriophage DNA into phage particles that become infectious, and
(5) Lysing the cell to release infectious particles that can find new hosts.

It has now been found that phage/host bacterium interaction can be measured in a fast and reliable way using techniques that measure phage proliferation in steps (3) and (5) of its cycle.

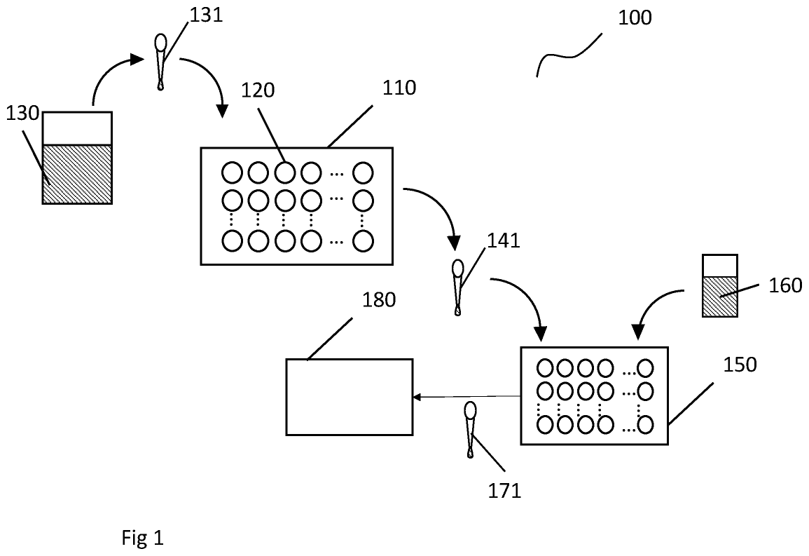

FIG. 1 schematically illustrates a system 100 and a method for the identification of a bacterium in a sample, for the identification of any phages that cause at least cell inactivation in said bacterium, and/or for the development of a composition of phages for the purpose of treating a bacterial infection caused by said bacterium, according to embodiments of the invention. As such, the system 100 and method described herein measure the sensitivity of a bacterium to a plurality of phages.

According to an aspect of the invention, there is provided a system 100. The system 100 comprises a microwell plate 110 comprising a plurality of wells 120, each well of said plurality of wells configured to contain one of a plurality of phages, wherein said microwell plate 110 is further configured to have a sample comprising a bacterium, preferably a pathogenic bacterium, dispensed into each well of said plurality of wells.

According to embodiments of the invention, the microwell plate 110 comprises at least 6 wells, preferably at least 12 wells, more preferably at least 24 wells, and even more preferably at least 48 wells. It is further understood that the microwell plate 110 can contain at most 9000 wells, preferably at most 5000 wells, more preferably at most 1000 wells, even more preferably at most 500 wells, and most preferably at most 200 wells. Typically, said microwell plate 110 will contain 96 wells, or thereabouts. The wells are usually arranged in a 2:3 rectangular matrix.

For the purpose of the invention, the term “microwell plate” is interchangeably used with the terms “microplate” or “microtiter plate”.

According to embodiments, the top of each well of the microplate 110 is sealed off for the purpose of retaining the well's content and for separating the well's content of the environment. Said seal (not shown on the figure) may be an individual seal for each single well. Preferably, one seal is commonly used to seal off all wells of said plurality of wells 120.

Preferably, the wells are sealed by one transparent polymer sheet, said sheet being perforable e.g. by the tips of disposable pipettes which are used by a liquid dispensing robot.

Microplates 110 are preferably made of or contain polymer materials, such a polycarbonate, polypropylene or polystyrene, although any material which is substantially inert to reactions with the contents of the well may be used. The microplate may be coloured, such as black or white, to facilitate measuring luminescence or fluorescence. Microplates may also comprise different structural elements, at least one of which is made of or contains one of the aforementioned materials. A 96-well microplate will typically have a working volume of 100 μl-360 μl.

According to preferred embodiments of the invention, the wells of the microplate 110 are configured to contain one type or species of phage for each well.

The bacteriophages may be inserted by dispensing a liquid containing phages of one specific phage type into a well, which liquid is subsequently removed through evaporation or freeze drying, leaving the phage in a solid state on the bottom of the well.

The phages may also be retained in the well in a solid matrix, e.g. as being part of a carrier, e.g. a porous carrier. Regardless of the method used, the phages can be dissolved in a liquid medium, typically an aqueous medium containing a bacterial species of interest, in a period of seconds.

It is important that no notable cross-contamination occurs between wells, due to the interaction between a Bacterium species and a phage which has migrated from a neighbouring well.

According to preferred embodiments, each well of the microplate 110 contains at most one type of phage, which phages have been selected due to their capacity to affect a species of the Bacteria domain. Advantageously, this allows to identify the presence of a certain species of Bacterium by administering a sample containing said species to all occupied wells and observing the effect of the selected phages on the Bacterial species as well as observe the degree of sensitivity of the bacterium to any of the phages of the plurality of phages present on the microplate 110.

A Bacterial species may come in the form of one or more of different strains of said species. Phages interacting with a Bacterium species are known to affect strains thereof to a different degree. By measuring the phage-bacterial host interaction for a selected group of phages that are known to affect said species, one may advantageously derive which phage(s) are more successful in lysing the administered cells. Moreover, testing a variety of phages which are each known to have a possible effect on a particular Bacterial species, allows for selecting at least one phage which may have the most pronounced effect on said strain for use in a treatment.

According to embodiments of the invention, a microplate 110 contains at least one selection of phages, wherein a selection of phages refers to a group of two or more phages which have the capacity to affect at least one strain of a specific Bacterial pathogen species.

Preferably, said selection is as complete as possible so that as many as possible strains of a single Bacterial species, and mutants thereof, can be identified as sensitive through interaction with at least one bacteriophage of the selection.

Preferably, a selection of bacteriophages will be as complete as possible so that a maximal number of host receptors, meaning binding receptors, of the specific Bacterial pathogen species is targeted.

According to embodiments of the invention, the number of phages may be optimized and matched to the number of Bacteria colony-forming units (CFU) in the sample. Indeed, if the number of phages is too low in the well relative to the number of target bacteria cells, the test will require a lower than necessary Limit of Quantification to function as fewer cells get infected and less replication occurs. However, if the phage concentration in the well is too high relative to the concentration of target bacteria, the progeny phages that are to be detected might be swamped by the number of phages added to the sample chamber, requiring a higher than necessary sensitivity. Preferably, regardless the type or species of phage, the number of phages is essentially the same in each well of the microplate 110, with deviations to the number average of phages in all wells not exceeding 20%, preferably 15%, more preferably 10%, even more preferably 5% and most preferably 2%. Preferably, the number of phages is the same for each well of the microplate 110.

According to embodiments of the invention, a well of the microplate 110 will contain a phage having a number of at least 10⁶ plaque-forming units (PFU), preferably at least 10⁷ PFU, and at most 10¹¹ PFU, preferably at most 10¹⁰ PFU and more preferably at most 10⁹ PFU. Typically, for phages occupying only a single well of the microplate 110, a well will contain around 10⁸ PFU phages.

According to embodiments of the invention, the microplate 110 contains one selection of phages, targeting the strains of a single Bacterial species. Alternatively, the microplate 110 contains two, three or four or more selections of phages, targeting two, three or four or more different Bacterial species, respectively.

Advantageously, this allows to identify the presence of at least two, three or four or more different Bacterial species.

The use of two, three, four or more selections of phages on a microplate may allow for the putative simultaneous identification of several bacterial species and test the sensitivity of these species to a plurality of phages.

As such, a native (possibly mixed) sample may be tested. Interaction measured between a phage and a native sample may then be presumed to faithfully indicate the presence of bacteria of interest.

In embodiments according to the invention, the identification of any phages that cause cell inactivation in the bacterial pathogen of interest, may exceed the capacity of a single microplate 110. In such cases, an array or plurality of microplates 110 is used.

Preferably, a selection of bacteriophages physically occupies a set of neighbouring wells on the microplate 110.

As a consequence of the above, the Phagogram may advantageously divulge at a glance the putative identity of a Bacterial species, as well as the sensitivity of the species to a plurality of phages.

According to embodiments, said microwell plate 110 is a pre-prepared plate comprising a plurality of phages and having a protection sealing off said wells. Said pre-prepared plate is configured to be introduced in a typical laboratory robot, such as a liquid dispensing robot, wherein samples containing a bacterial species can be distributed over and dispensed in said wells, thereby puncturing said seal. Said pre-prepared plate is typically a disposable microwell plate.

It has been found that such a pre-prepared microwell plate, when kept at a temperature of 20° C. and a humidity of 50%, can be preserved for a period of at least 12 months.

According to preferred embodiments of the invention, at least one well of said plurality of wells will be filled with a “blank” composition, wherein said blank composition does not contain any concentration of phages of a specific type which is higher than a background concentration. Advantageously, by comparing measurements of the blank composition and the other phage-containing wells, the effect of phages on a bacterium species can be studied.

According to an aspect of the invention, there is provided a method for measuring the sensitivity of a bacterium to a plurality of phages.

The method comprises the following steps:
Providing a first composition 130 comprising said bacterium and a first microwell plate 110 comprising a plurality of wells 120, each of said plurality of wells 120 containing one of said plurality of phages;
Dispensing a first sample 131 of said first composition 130 in at least one well of said plurality of wells 120;
For each of said at least one well, allowing incubation of the mixture of said first sample 131 comprising said bacterium with a bacteriophage contained in the well for the duration of a first period of time;
Optionally, transferring a second sample 141 of said mixture to a second microwell plate 150,
Adding at least one reagent and/or additive 160 for each mixture on said first 110 or second microwell plate 150,
Transporting to and introducing at least one mixture or a final sample 171 of said at least one mixture, after receiving said at least one reagent and/or additive 160, in an analytical device 180 and measuring the bacterium-phage interaction of said final sample by said analytical device; and
Interpreting and presenting the measurements by said analytical device 180 in the form of a phagogram by a control unit (not shown on the figure).

According to embodiments of the invention, the first composition 130 comprises at least one bacterial species of interest which may be a bacterial pathogen or infectious agent.

According to preferred embodiments of the invention, said first composition 130 will refer to or contain a patient sample, being a native sample from a patient, containing said bacterial pathogen. It will be understood that said bacterial species refers herein to a species or Genus of interest of the Bacterium domain, which is subject to identification by said method and for which a treatment may be sought. The patient sample may and most likely will include other species of Bacteria, but due to the high specificity in bacteriophage/host Bacterium interaction, these other species will not interfere in the detection of possible phage expression.

According to alternative embodiments of the invention, said first composition 130 refers to or contains a pure culture isolate of a patient's native sample.

Independent of whether a native sample or a pure culture isolate is used in the system and method described herein, the first composition 130 will further contain at least a buffer, such as a McFarlan buffer or any other suitable buffer known in the state of the art.

Typically, the first composition 130 will have a volume of at least 200 ml.

A sample of the bacterial species may be obtained from a patient, being a human or an animal. Said animal can be cattle or farm animals. Typically, the sample will be obtained from a patient, suspected of having a bacterial infection.

The sample may include a cell, tissue, biopsy, secretion or exudate, fluid, or gastric contents obtained from the subject. Examples of samples include, but are not limited to, blood, lymph, urine, a skin scrape or swab, nasal secretion, ear wax, serum, a surface washing, plasma, cerebrospinal fluid, saliva, sputum, stool, vomitus, milk, tears, sweat, biopsied tissue, bedding, litter, or egg wash.

The sample may however also be obtained from the environment, in particular in cases where it is suspected that parts of the environment are contaminated by bacteria, which may further infect living beings. In alternative embodiments, the sample is obtained from an environmental locus, such as a food supply, water source, area of soil, or a building.

Given that the origin of the sample may vary greatly, the concentration of the Bacterial species of interest in the sample will vary as a consequence. It has been found that for a native sample to be used directly in the first composition 130, without isolation of the Bacterial species of interest, a concentration of at least 10⁵ CFU/ml, preferably at least 10⁶ CFU/ml, and more preferably of least 10⁷ CFU/ml is required. Such minimal concentration is however subject to the overall quality of the sample.

In preferred embodiments according to the invention, the first composition 130 contains a native patient sample, wherein the concentration of the Bacterial species of interest in the composition 130 is at least 10⁵ CFU/ml, preferably at least 10⁶ CFU/ml, and more preferably of least 10⁷ CFU/ml.

In these embodiments, there is no need for isolating the Bacterial species of interest and subsequently for growing said species to a predetermined concentration which is required by the detection limit of said analysis techniques. This allows for the rapid analysis of an infectious bacterium in a patient sample, going from identification of the particular Bacterial strain in the patient sample to identification of at least one bacteriophage affecting the Bacterial population. Identification of such a bacteriophage may then lead to a treatment for the patient involved. The detection of the reactive phages at the same time precisely determines the bacterium species responsible for the infection.

Alternatively, a pure culture isolate of the Bacterial species is first obtained, which may be subsequently grown to a predetermined concentration. This is typically done by amplifying the bacteria species of interest on a suitable medium.

In these alternative embodiments according to the invention, the first composition 130 contains a pure isolate obtained from a native patient sample, wherein the concentration of the Bacterial species of interest in the composition 130 is at least 10⁵ CFU/ml, preferably at least 10⁶ CFU/ml, and more preferably of least 10⁷ CFU/ml.

In a step of the method according to the invention, a first sample 131 of said first composition 130 is dispensed in at least one well of said plurality of wells 120 of the microplate 110. Preferably, the microplate 110 is organized as described herein above.

The bacteria and the phages are preferably mixed in a liquid medium in said well. The liquid for providing the liquid medium is typically originating from the first sample 131, comprising at least the bacterial species of interest and the buffer. By not using a gel, the liquid medium advantageously facilitates transfer of samples of said mixture to a device for further analysis.

According to preferred embodiments of the invention, the liquid medium is selected so as to be capable of essentially dissolving the complete bacteriophage population in a time span of a couple of seconds.

According to the invention, the phage is located in the well and a first sample 131 containing the bacterium species is added to the phage in the well.

In a step of the method according to the invention, said first sample 131 comprising said bacterial pathogen mixed with a bacteriophage contained in the well is allowed to incubate for the duration of a first period of time.

In embodiments according to the invention, said first period of time is at least 30 minutes, preferably at least 40 minutes and more preferably at least 50 minutes. Said first period of time is preferably not longer than 2 hours, more preferably not longer than 90 minutes. Typically, said first period of time is around 1 hour. It has been found that this period of time allows the bacteriophage to connect to the binding receptors of the Bacterium species, in the case such interaction between both is possible.

During said first period of time, mixing the phages and the bacterial species of interest occurs at a temperature of at least 30° C., preferably at least 33° C., and most preferably at least 36° C. It is further understood that mixing both components occurs at a temperature of at most 40° C., preferably at most 39° C., and most preferably at most 38° C. Typically, the phages and the bacterial species of interest are mixed in the wells at a temperature of around 37° C.

In a subsequent step, second samples 141 of said mixtures are transported for measurement into an analytical device 180 for further characterization.

Optionally, for each well, a second sample 141 containing the mixture of bacteriophage and Bacterial species is transferred to a second microwell plate 150, said second microwell plate 150 being configured for introducing said mixtures into said analytical device 180, so that samples in its wells can be measured by the device 180. Said second microwell plate 150 is thereby configured to be compatible with the analytical device 180, which may often require smaller volumes of samples. Transfer of second samples 141 of said first microplate 110 to said second microplate 150 is typically done by use of a liquid pipetting robot. The analytical device 180 may then, in a step of the method according to the invention, analyse and measure the samples on the second microplate 150. In a subsequent step of the method according to the invention, a computing means or control unit may then dispose of the measurements provided by the device 180 and provide for an interpretation and presentation of the measurements in the form of a Phagogram.

In embodiments of the system and method according to the invention, the computing means or control unit is configured to steer the parts of the system and the steps of the method provided herein, so that said Phagogram may be obtained in an automated way.

The control unit may be or may contain a controller for steering, evaluating, and processing the steps of the method, such as transfer of samples between microplates, controlling temperature of the mixtures and the microplates, controlling the adding of additional reagents and/or additives 160, the analysis of the samples or mixtures by the analytical device 180, and/or obtaining the results of the measurements in the form of a Phagogram.

In embodiments according to the invention, the control unit may be furthermore configured to communicate the results of said phagogram to a central server, thereby advantageously allowing sharing the phagogram with a plurality of system users.

In preferred embodiments according to the invention, further reagents and/or additives 160 are added to the phage/bacterium mixtures prior to measurement on said analytical device 180. The time of administration prior to measurement, nature and number of such at least one reagent and/or additive 160 depend on the analysis technique that is chosen. Said at least one reagent and/or additive 160 may be added to the phage/bacterium mixtures when located on the second microplate 150.

Alternatively, the mixtures in the wells of the first microplate 110 are analysed by the analytical device 180. Any at least one reagent and/or additive 160 are added to the phage/bacterium mixtures situated in the wells of the first microplate 110, which is configured to be compatible with the device 180, so that samples in its wells can be measured by the device 180.

In embodiments according to the invention, said further analysis comprises at least one of:
Measuring the abundance of phage DNA in the second sample 141 by use of qPCR and fluorescence;
Detecting cell lysis by measuring bioluminescence through addition of luciferase.

The present invention is based inter alia on the insights of the inventors that both of said techniques are capable of measuring and identifying interaction between the phage and the host bacterium cell in a patient sample within a reasonable amount of time, allowing ultimately for the selection of an appropriate phage or a combination of phages suitable for the treatment of a patient affected by said bacterium.

It has been found that both reliably measure bacteriophage/host bacterium interaction. Typically, only one of said two techniques will be used at a time.

In embodiments according to the invention, said at least one analytical device 180 is configured to measure the interaction of each of said plurality of phages with said bacterial pathogen by measuring the concentration gradient of phage DNA by use of quantitative polymerase chain reaction (qPCR). Phage/host bacterium interaction is thus evaluated by use of qPCR, which is a well-known laboratory technique based on the Polymerase Chain Reaction.

The PCR process generally consists of a series of steps, including
(1) Denaturation, consisting of heating the sample to a temperature of about 94° C.-96° C., allowing the hydrogen bonds of double stranded DNA in the sample to break, yielding two single-stranded DNA molecules;
(2) Annealing, by lowering the temperature in the sample to about 50° C.-65° C., allowing the binding of single-stranded primers to each of the single-stranded DNA molecules. Typically, two different primers are used, each complementing a target region of the single-stranded DNA molecules, at the 3′ end of each strand; and
(3) Elongation, by having a DNA polymerase synthesize a new DNA double strand by adding free dNTPs (nucleoside triphosphates containing deoxyribose) from the reaction mixture.

Under optimal conditions, the number of DNA sequences is doubled at each elongation step. With each successive cycle, the original template strands plus all newly generated strands become template strands for the next round of elongation, leading to exponential amplification of the specific DNA target region. The processes of denaturation, annealing and elongation constitute a single cycle. Multiple cycles are required to amplify the DNA target to millions of copies.

While the PCR process offers a fast and inexpensive method to obtain DNA amplification, qPCR or real-time PCR has the advantage that it can measure and follow the amplification process in real time, hence measuring the concentration gradient of DNA, by use of fluorescence spectroscopy. Fluorescence spectroscopy or fluorimetry refers herein to an analytical technique to detect and analyse the fluorescence in the sample.

In embodiments according to the invention, molecular beacon probes are used for measuring concentration of bacteriophages in a phage—host bacterium mixture by measuring the fluorescence in said mixture.

In specific embodiments according to the invention, such molecular beacon probes are TaqMan probes.

The molecular beacon probes used herein consist of a fluorophore covalently attached to the 5′-end of the probe and a quencher at the 3′-end. As long as the fluorophore and the quencher are in proximity, the quencher molecule quenches the fluorescence emitted by the fluorophore.

The molecular beacon probes used herein are typically designed so that they anneal within a specific region of a single-stranded DNA molecule that is complementary to the probe sequence, amplified by a specific set of primers.

As the polymerase extends the primer in the elongation step and synthesizes the nascent double strand, the molecular beacon probe that has annealed is degraded, thereby releasing the fluorophore and breaking the proximity between the fluorophore and the quencher. Hence, fluorescence is detected which is directly proportional to the amount of fluorophores released. The resulting fluorescence signal permits quantitative measurements of the accumulation of the bacteriophage DNA material.

It is inherent to the qPCR process that the primers, nucleic acid polymerase and molecular probes are selected in view of the bacteriophage which is mixed with the Bacterium host. Prior to analysis by the analytical device 180, each specific mixture of bacteriophage—bacterium host in the wells of the first microplate 110 or second microplate 150 can therefore be provided by a specific selective composition, containing at least one of primers, nucleic acid polymerase and molecular probes which are related to the specific bacteriophage that is present in a well of the microplate 110 or in the mixture.

According to embodiments of the invention, said at least one reagent and/or additive 160 that is added to the sample prior to analysis by the analytical device 180 comprise at least one of the following:
at least one primer molecule;
DNA polymerase;
dNTPs;
a molecular beacon probe, capable to fluoresce; a buffer;
qPCR-amplification compatible solvents and enzymes known in the state of the art.

Said at least one reagent and/or additives 160 may be specific for a single combination of bacteriophage—host bacterium. Typically, said at least one reagent and/or additives 160 may be specific for the bacteriophage in the mixture.

The inventors have found that by use of qPCR, a differentiation can be made between phages that can and cannot productively infect the bacterial species of interest which may be present in samples obtained from a patient. This is done by directly measuring the abundance of phage DNA within infected cells. In other words, the replication of phage DNA within infected cells—or lack thereof—is used as a benchmark and point of comparison for evaluating the capacities of a phage to infect a bacterium of interest. Such comparison is then made with the still encapsulated DNA surrounding uninfected cells and negative controls in other phage-Bacterium mixtures.

The present invention is based on the insight that, due to its extraordinary sensitivity, qPCR allows the measurement of even small changes in target abundance, which allows the detection of even relatively small changes in the abundance of phage DNA caused by replication. Advantageously, the target specificity allows the qPCR technique to be used directly on patient samples rather than pure culture isolates, saving significant time.

In the embodiments described hereabove, the analytical device 180 may then refer to a qPCR analytical tool or instrument, adapted for measuring the differential concentration of phage DNA in real-time. Advantageously, such instrument is configured for evaluating and measuring in parallel, meaning that the concentration gradient of phage DNA of several wells can be obtained simultaneously. Typically, the instrument is configured to measure the fluorescence in the wells of a 96-well microplate.

In embodiments according to the invention, said at least one analytical device 180 is configured to measure the interaction of each of said plurality of phages with said bacterium or bacterial pathogen by detecting cell lysis of said pathogen through measurements of bioluminescence which is caused by the addition of a Luciferase/Luciferin complex.

Luciferase is used in a variety of applications. Assays for determining the presence or absence of microbes by using Luciferase-mediated oxidation of bacterial ATP have existed since long. They are currently in use in a variety of diagnostics as well as commercially available kits for detecting bacterial contamination.

The reaction catalyzed by luciferase enzyme and emitting light, is the following (formula (1)):

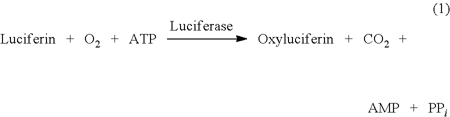

wherein PP, relates to pyrophosphate. In the luciferase reaction, light is emitted when luciferase acts on the appropriate luciferin substrate. Photon emission can be detected by light sensitive apparatus such as a luminometer or modified optical microscopes. The luciferase reaction allows observing biological processes.

The present invention is based on the existing insight that Luciferase can act as an ATP sensor protein. Luciferase is hence used to detect the efflux of ATP from a lysed cell thereby effectively displaying the real-time release of ATP through bioluminescence. Powered by ATP to produce bioluminescence, luciferases allow for the very sensitive detection of the amount of ATP in solution in even very low concentrations.

Most healthy bacterial cells contain around 2×10⁻¹⁸ mol ATP per cell, and commercially available kits and analytical tools typically have a detection limit between 1×10⁻¹⁷ mol ATP and 1×10⁻¹³ mol ATP. As a consequence, an assay based on this principle may expect to detect down to a range between about 5 and about 50,000 healthy bacterial cells. The inventors have found that even at the high end of the range of detection limit, a test based on the principle of measuring bioluminescence caused by a combination of a compatible Luciferin and Luciferase, reliably detects the ATP released by the lysis of 2% of the viable cells available in the least abundant pure culture isolates.

It has been found that the Luciferin/Luciferase combination is not specific to the bacteriophage used. Hence, a suitable Luciferin/Luciferase combination may be used for all bacteriophage—host bacterium mixtures.

It has further advantageously been found that high concentrations of the Luciferin/Luciferase combination do not affect the bioluminescence measurements.

According to embodiments of the invention, said at least one reagent and/or additives 160 that is added to the sample prior to analysis by the analytical device 180 comprises at least one of the following:
a Luciferin/Luciferase combination or complex being adapted to allow detection of bacterial cell lysis through depletion of ATP of the lysed cells by the Luciferin/Luciferase combination or complex while concomitantly emitting light;
Luciferin/Luciferase complex compatible solvents and enzymes known in the state of the art.

In the embodiments described hereabove, the analytical device 180 may then refer to a light sensitive apparatus such as a luminometer or an optical microscope, adapted for measuring luminescence emitted by the reaction described in formula (1).

In preferred embodiments according to the invention, such instrument is configured for evaluating and measuring in parallel, meaning that the bioluminescence of mixtures of several wells can be measured simultaneously.

The instrument may be configured to measure the bioluminescence in the wells of a 96-well microplate. Alternatively, the instrument is configured for measuring sequentially the mixtures.

In embodiments of the method according to the present invention, the method further comprises the step of selecting a composition of phages on the basis of the phagogram, presented by the control unit, wherein the phages of said composition of phages are selected due to the ability to interact with a maximal number of different binding receptor types of said bacterium.

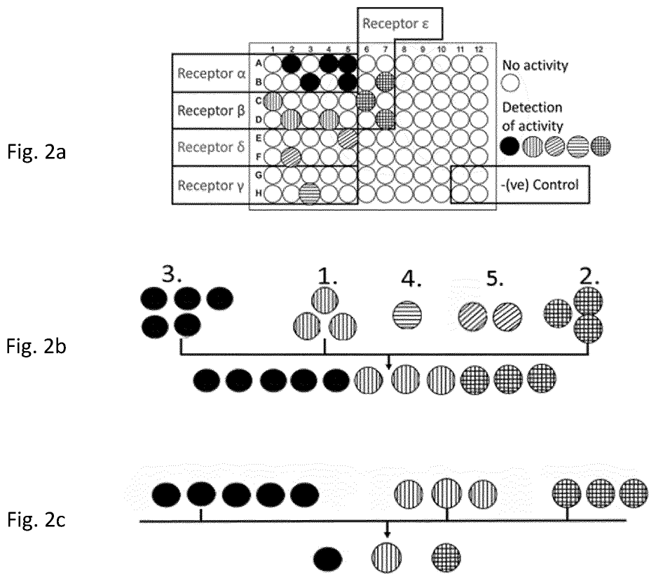

FIG. 2a-2c schematically illustrate these embodiments.

According to these embodiments, a goal of the method is to propose a composition or combination of phage types on the basis of a phagogram.

Preferably, such a composition will consist of one to three phages, wherein each of these one to three phages target a receptor type or receptor family, preferably different receptor types. A combination of phage types may be used as a basis for a treatment of an infection caused by said bacterium.

According to embodiments, the control unit is configured to execute the steps of the method hereabove based on the results of the measurements provided by said at least one analytical device in the form of a Phagogram using the system and following the method as described here above.

The control unit will interpret the obtained results by assigning phages to receptor groups, wherein phages in a receptor group interact with a bacterium via a specific binding receptor type, and by ranking said phages in the group on the basis of successful interaction with the bacterium species.

Reference is made to FIGS. 2a-2b. In a particular case, 14 phages that recognize five different receptor families or binding receptor types of a bacterial species of interest are found to have activity against the species. Five phages are found that target the α receptor family of the bacterial species, three phages that target the β receptor family, two phages that target the δ receptor family, one phage that targets the γ receptor family, and three phages that target the ε receptor family are identified as being appropriate for having a maximum interaction with the bacterium and for combatting an infection caused by the bacterium.

Subsequently, for each receptor family, the control unit is configured to select a single phage from each receptor group that is most appropriate for targeting the related receptor family and to identify the best phage within that group.

In order to generate a cocktail of three phages, three receptor families to target need to be selected. This will be done by ranking each receptor family target from most to least for each possible indication with antibiotic sensitivity and without antibiotic sensitivity. Referring to FIG. 2b, the a receptor family, the β receptor family and the ε receptor family are withheld.

For selecting a phage within a receptor group, and referring to FIG. 2c, the control unit will use the information provided by the phagogram and will typically select a phage which has shown the highest interaction with the bacterial species, i.e. that phage is selected for which the bacterium has shown the highest sensitivity. This process allows for the automated proposal of a cocktail for a physician to prescribe that will be tailored to the susceptibility of the patient's bacterial strain as well as the clinical context of their infection.

While the invention has been described hereinabove with reference to specific embodiments, this is done to illustrate and not to limit the invention, the scope of which is defined by the accompanying claims. The skilled person will readily appreciate that different combinations of features than those described herein are possible without departing from the scope of the claimed invention.

1. A system (100) for measuring the sensitivity of a bacterium to a plurality of phages, the system (100) comprising:
A microwell plate (110) comprising a plurality of wells (120), wherein each of said plurality of wells (120) is configured to contain one of a plurality of phages, wherein said microwell plate (110) is further configured to have a sample comprising said bacterium dispensed into each of said plurality of wells (120);
at least one reagent and/or additive (160), configured to be added to at least one mixture of said sample comprising said bacterium with a phage of said plurality of phages; and
A control unit configured to interpret and present the results of measurements in the form of a phagogram, said measurements being provided by at least one analytical device (180), said at least one analytical device (180) being configured to measure the interaction of each of said plurality of phages with said bacterium;
wherein said at least one reagent and/or additive (160) comprises at least one of the following: a primer molecule, a DNA polymerase, a nucleoside triphosphate (dNTP), a molecular beacon probe capable to fluoresce, a buffer, and a qPCR-amplification compatible solvent and/or enzyme; and
wherein said at least one analytical device (180) is further configured to measure the interaction of each of said plurality of phages with said bacterium by measuring the abundance and/or differential concentration of phage DNA by use of quantitative polymerase chain reaction;
or,
wherein said at least one reagent and/or additive (160) comprises at least one of the following: a Luciferin/Luciferase complex configured to allow detection of bacterial cell lysis through depletion of ATP and concomitant fluorescein light emission, and a Luciferin/Luciferase complex compatible solvent and/or enzyme, and
wherein said at least one analytical device (180) is further configured to measure the interaction of each of said plurality of phages with said bacterium by measuring said light emission.

2. The system according to claim 1, wherein said sample is a native sample from a patient.

3. The system according to claim 1 or claim 2, wherein said microwell plate (110) is disposable.

4. The system according to any one of the preceding claims, wherein said plurality of wells (120) is further configured to contain at least one selection of phages, wherein each of said selection of phages is selected by the capacity to interact with and/or inactivate a single specific bacterial species, and strains thereof.

5. The system according to any one of the preceding claims, wherein said control unit is furthermore configured to provide a composition of phages on the basis of said phagogram, wherein said composition of phages interact with a maximal number of different binding receptor types of said bacterium.

6. A method for measuring the sensitivity of a bacterium to a plurality of phages, the method having the following steps:
Providing a first composition (130) comprising said bacterium and a first microwell plate (110) comprising a plurality of wells (120), each of said plurality of wells (120) containing one of said plurality of phages;
Dispensing a first sample (131) of said first composition (130) in at least one well of said plurality of wells (120);
For each of said at least one well, allowing incubation of the mixture of said first sample (131) comprising said bacterium and a bacteriophage contained in the well for the duration of a first period of time;
Adding at least one reagent and/or additive (160) to each mixture;
Transporting to and introducing at least one mixture in an analytical device (180) and measuring the bacterium-phage interaction of said at least one mixture by said analytical device (180); and
Interpreting and presenting the measurements by said analytical device (180) in the form of a phagogram by a control unit.

7. The method according to claim 6, wherein the method further comprises the step of transferring a second sample (141) of said mixture to a second microwell plate (150), prior to adding said at least one reagent, wherein said second microwell plate is configured to be compatible with the analytical device (180).

8. The method according to claim 6 or claim 7, wherein said first composition (130) is a native sample from a patient.

9. The method according to any of claims 6-8, wherein measuring the bacterium-phage interaction by said analytical device (180) implies measuring the abundance and/or differential concentration of phage DNA by use of quantitative polymerase chain reaction.

10. The method according to any of claims 6-8, wherein measuring the bacterium-phage interaction by said analytical device (180) implies detecting cell lysis by measuring bioluminescence through addition of luciferase.

11. The method according to any of claims 6-10, wherein the method further comprises the step of selecting a composition of phages on the basis of the phagogram, wherein the phages of said composition of phages are selected due to the ability to interact with a maximal number of different binding receptor types of said bacterium.

12. A microwell plate (110) comprising a plurality of wells (120) for each containing one of a plurality of phages, for use in the system according to claims 1-5 and method according to claims 6-11.

13. A computer program product comprising code means configured to cause a processor of a control unit to perform the functions of said system according to claims 1-5 and method according to claims 6-11.
